(19) United States
(12) Patent Application Publication
(10) Pub. No.: US 2025/0052769 A1
(43) Pub. Date: Feb. 13, 2025

(54) MIMOTOPES OF ARA H 2 AND USES OF THE SAME TO DIAGNOSE PEANUT ALLERGIES

(71) Applicant: The Regents of the University of Colorado, A Body Corporate, Denver, CO (US)

(72) Inventors: Stephen Dreskin, Denver, CO (US); Catherine Braun-Schein, Galveston, TX (US); Xueni Chen, Denver, CO (US)

(21) Appl. No.: 18/787,548

(22) Filed: Jul. 29, 2024

Related U.S. Application Data
(60) Provisional application No. 63/516,204, filed on Jul. 28, 2023.

(51) Int. Cl. G01N 33/68; C07K 14/415

(52) U.S. Cl. CPC G01N 33/6893; C07K 14/415; G01N 2800/24

(57) ABSTRACT

Disclosed herein are compositions and methods for predicting or assessing the presence and/or severity of IgE-mediated peanut allergy in a subject using peptides and or mimotopes that bind as well or better than naturally occurring sequences. The disclosed methods and compositions may comprise one or more peptides or mimotopes of Ara h 2, specifically Epitope 3, or a portion thereof. In many embodiments, the peptides and mimotopes may comprise at least one DPY_P sequence, and in many cases the second proline, “P,” in the sequence may be hydroxylated (i.e. “h”) and may be represented by h. In many embodiments, substituting a P->h within the disclosed peptide and mimotope sequences may increase binding of IgE from the subject. In many embodiments, enhanced binding of IgE from a subject indicates that the subject suffers from an IgE-mediated peanut allergy.

### CROSS-REFERENCE TO RELATED APPLICATIONS

This application claims benefit of priority pursuant to 35 U.S.C. § 119(e) of U.S. provisional patent application No. 61/516,204 entitled “MIMOTOPES OF ARA H 2 AND USES OF THE SAME TO DIAGNOSE PEANUT ALLERGIES,” filed on 28 Jul. 2023, which is hereby incorporated by reference in its entirety.

### BACKGROUND

IgE-mediated peanut allergy is a major health concern affecting approximately 2% of children and adults in the US, the UK and Europe. Recent progress with early administration of peanuts (PN) and other allergenic foods should reduce the number of new cases, but there are significant limitations with implementation. Oral immunotherapy (OIT) for PN allergy, but not yet tree nut (TN) allergy, is currently approved. Sublingual as well as epicutaneous immunotherapy for PN allergy hold promise. OIT has limitations including cost, compliance, lack of efficacy and unpredictable breakthrough.

There is an urgent need for new methods for analyzing a subject having or at risk of an IgE-associated sensitivity to peanut allergens—especially Ara h 2 and specific epitopes thereof.

### SEQUENCE LISTING

The present disclosure further incorporates by reference the Sequence Listing submitted herewith. The Sequence Listing .xml file, identified as file name P310144US02.xml, is 118,122 bytes in size and was created on Sep. 27, 2024. The Sequence Listing, electronically filed herewith, does not extend beyond the scope of the specification, and does not contain new matter.

### SUMMARY

Disclosed herein are compositions, methods, and systems for determining a patient with a peanut allergy and/or severity of same. The disclosed compositions may include one or more peptide or mimotope of a peanut 2S albumin, for example Ara h 2, in some embodiments Epitope 3 of Ara h 2. In many cases, the disclosed peptides may comprise a substitution of proline for hydroxyproline, which may increase binding of serum IgE to the peptide. In many embodiments, the disclosed peptides and mimotopes may be used to determine whether a subject suffers from a peanut allergy and/or severity of such allergy. In some embodiments, the disclosed peptides may competitively bind IgE in the serum of a patient and block or reduce IgE binding to a peanut 2S albumin, for example Ara h 2, in various embodiments Epitope 3 or Ara h 2.

In some embodiments, peptides having specific sequences are disclosed, in many embodiments the peptides may form linear mimotopes of a conformational epitope of a 2S albumin of a peanut. In some embodiments, the peptides may have amino acid sequences selected from SEQ ID NO: 1-66, for example SEQ ID NO: 2 (See Table 3). In some embodiments, the peptide may have an amino acid sequence selected from SEQ ID NO: 2-10, 12-25, and 47-66, and IgE may bind the peptide with higher affinity where a hydroxyproline is replaced with proline. In some embodiments. Also disclosed is non-native peptide having the sequence of SEQ ID NO: 11, wherein more IgE binds the non-native peptide than to the respective wild-type peptide.

Also disclosed are methods of diagnosing a peanut allergy in a subject, the method comprising: contacting serum from the subject with one or more peptides, and measuring binding of IgE in the serum to the one or more peptides, wherein the one or more peptides has an amino acid sequence selected from SEQ ID NO: 1-66, and wherein the peptide binds more IgE from serum of a subject with peanut allergy than from serum of a subject without peanut allergy. In some embodiments, the one or more peptides has the sequence of SEQ ID NO: 2 and/or the peptides are presented in an immunoassay. In many embodiments, the native form of the peptide may bind fewer IgE than a non-native form, for example wherein at least one proline is hydroxylated.

Also disclosed are methods of predicting clinical severity of a peanut allergy in a subject, the method comprising: contacting serum from the subject with one or more peptides, and measuring binding of IgE in the serum to the one or more peptides, wherein the one or more peptides has a sequence selected from SEQ ID NO: 1-66, and wherein binding of IgE to the one or more peptides at a statistically significantly greater amount than binding of IgE from a known allergic person to the one or more peptides indicates increased sensitivity to peanut allergens in the subject. In some embodiments, the one or more peptides has the sequence of SEQ ID NO: 2 and/or the peptides may be part of a microarray.

Also disclosed are methods of predicting the outcome of an immunotherapy to treat a peanut allergy in a first subject, the method comprising: contacting serum from the first subject with one or more peptides, and measuring binding of IgE in the serum to the one or more peptides, wherein the one or more peptides has an amino acid sequence selected from SEQ ID NO: 1-66, and wherein binding of IgE to the one or more peptides in the first subject at a level greater than binding of IgE from a second subject known to have a peanut allergy indicates likelihood of unsuccessful immunotherapy in the first subject. In some embodiments, the one or more peptides has an amino acid sequence of SEQ ID NO: 2 and/or the immunotherapy is selected from oral, subcutaneous, sublingual, epicutaneous, and intra-lymph node immunotherapy. In many embodiments, successful immunotherapy comprises desensitization of the subject to peanuts, or sustained unresponsiveness to peanuts.

Also disclosed are methods of affecting IgE binding in a subject, the method comprising: contacting serum from the subject with a peptide having an amino acid sequence of SEQ ID NO: 2, and allowing the peptide to interact with IgE in the serum, wherein the peptide interferes with binding of IgE to at least one peptide having a native sequence comprising a DPYSP motif or a DPYSh motif. In some embodiments, the peptide at least partially inhibits IgE binding to an Ara h 2 molecule.

Also disclosed are peptides, having a sequence selected from SEQ ID NO: 1-66, that may form mimotopes, for example linear mimotopes, of a conformational epitope of a 2S albumin of a peanut, in some embodiments, the peptide may have amino acid sequence SEQ ID NO: 2. In many embodiments, the peptide may bind serum IgE and have an amino acid sequence selected from SEQ ID NO: 2-10, 12-25, and 47-66, and substitution of hydroxyproline for proline may increase the amount of serum IgE that may bind the peptide. In some embodiments, the peptide has amino acid sequence SEQ ID NO: 11, is a conformational mimotope of an epitope of a peanut Ara h 2, and wherein the peptide binds more serum IgE than a peptide derived from wild-type Ara h 2.

Also disclosed are methods of analyzing peanut IgEs in the serum of a subject, the method comprising: contacting serum from the subject with one or more peptides having an amino acid sequence selected from SEQ ID NO: 1-66; measuring the amount of serum IgE bound to the one or more peptides to generate a subject binding level; comparing the subject binding level to a first control binding level for an allergic patient, and and a second control binding level for a non-allergic patient; and thereby analyzing peanut IgEs in the serum of the subject. In some embodiments, the subject may be identified as having a peanut allergy where the subject binding level is equal or greater than the first control binding level. In some embodiments, the one or more peptides has amino acid sequence of SEQ ID NO: 2. In some embodiments, the contacting or measuring steps involve an immunoassay or a microarray. In some embodiments, wherein the subject biding level is greater than a binding a binding level measured using a wild-type peptide sequence. In some embodiments, the subject may have an increased sensitivity to peanut allergens wherein subject binding level is statistically significantly greater than the first control binding level.

Also disclosed are methods of affecting IgE binding in a subject, the method comprising: contacting serum from the subject with a peptide having the sequence of SEQ ID NO: 2, and allowing the peptide to interact with IgE in the serum, wherein the peptide interferes with binding of IgE to at least one peptide having a native sequence comprising a DPYSP motif or a DPYSh motif. In some embodiments, the peptide at least partially inhibits IgE binding to an Ara h 2 molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B. Alanine scanning of Epitope 3-Core with the serum pool. Microarrays containing alanine substituted peptides of epitope 3 were probed with the serum pool diluted to contain 10 IU/ml of Ara h 2-specific IgE. Results from a single assay (FIG. 1A; (SEQ ID NO:67, 74-89) show the net density units. Summary data from 3 assays with the density units normalized to the signal seen with the WT sequence are shown in (FIG. 1B; SEQ ID NO:67, 74-89).

FIGS. 2A and 2B. Alanine scanning of Epitope 3-Core with individual sera. Microarrays containing the alanine scan of epitope 3 were probed with each of 7 individual sera. Results are shown for single assays (FIG. 2A; Panels A-G; SEQ ID NO:67) with each serum. The bars show the net density units from microarray assay. To summarize data from 3-4 independent assays, the observed density units for each peptide within each assay were normalized to the signal seen with the WT sequence (FIG. 2B; Panels A-G; SEQ ID NO:67). Specific sera were D107 (Panel A), D197 (Panel B), D199 (Panel C), D213 (Panel D), D216 (Panel E), D217 (Panel F) and D218 (Panel G).

FIGS. 3A and 3B. A selected mimotope related to epitope 3; alanine scanning with the serum pool. Microarrays containing alanine substituted peptides of mimotope A147 were probed with the serum pool diluted to contain 1 IU/ml of Ara h 2-specific IgE. Results for mimotope A147 from a single assay (FIG. 3A; SEQ ID NO:2, 90-99) show the net density units. Summary data from 3 separate assays with the density units normalized to the signal seen with the original mimotope sequence is shown in (FIG. 3B; SEQ ID NO:2, 90-99).

FIGS. 4A and 4B. Model structure of Epitope 3-Core. FIG. 4A (SEQ ID NO:102) ribbon structure of original Ara h 2 peptide (green) overlaid with that of the peptide containing the P12A mutation (orange) shows the mutated side chain would move to the other side of the structure, away from the rest of the DPY_h motif. The orange arrow points to the orientation of the P12A substitution. The blue arrows point to the 3 residues of interest, ASP-8 (D8), TYR-10 (Y10) and PRO-12 (P12). As noted in the text, in the WT structure PRO-12 is h-12 due to post translational hydroxylation but this could not be accurately modeled with the available templates. FIG. 4B) Backbone overlay of all 16 alanine scanning models of the peptide, overlaid with A. The reference peptide structure is shown in green, peptide number 12 with P12A mutation (orange), and others are shown in gray color. Note that Ala mutations in the C-terminal of the peptide may cause more disorder in the structure than replacement in the first 7 residues.

FIGS. 5A and 5B. Identification of the sequence of the unstructured region of Ara h 2 used for alanine scanning. IgE binding by ELISA using the serum pool at 10 IU/ml of Ara h 2-sIgE. FIG. 5A (SEQ ID NO:67-70) Biotide® technology was used to generate 20-30% pure peptides of the unstructured region of Ara h 2 and truncated versions as shown. IgE binding with the serum pool was measured in triplicate. Peptides were added at 1 nM and Bt-anti-HuIgE at 1:3,000. A representative assay (mean±SD) of 3 independent assays is shown. FIG. 5B) 95-97% pure peptides of the 20mer unstructured region and of the optimum short version of this region, Epitope 3-Core, were synthesized. The 20mer version of epitope 3 (closed circles) and the optimized short version of epitope 3, Epitope 3-Core, (open circles) bound similar amounts of IgE. Peptides were added at 5 nM and Bt-anti-HuIgE at 1:500. Data from 3 independent assays were pooled and shown together (mean±SEM).

FIGS. 6A and 6B. Substitution of hydroxyproline for proline in mimotope A147 greatly enhances IgE binding and gives a 12mer peptide with similar IgE binding as the native sequence. IgE binding by ELISA using the serum pool at 10 IU/ml. FIG. 6A) Biotide® methodology was used to synthesize the 20mer peptide of the unstructured region (closed circles), 12mer mimotope A147 with the original sequence from the peptide library (closed triangles) and with hydroxyproline substituted for proline (P->h) (open triangles). Various amounts of peptide (1 nM) were added to wells coated with streptavidin and IgE binding of the serum pool (10 Iu/ml of Ara h 2-sIgE) was measured (Bt-anti-HuIgE at 1:3,000). The mimotope with P->h bound more IgE at all doses than did the original mimotope (p<0.01). A representative assay (mean±SD) of 3 independent assays is shown. FIG. 6B) Purified peptides of the mimotope with P->h and Epitope 3-Core were synthesized and assayed with ELISA for IgE binding with the serum pool. Peptides were added at 5 nM and Bt-anti-HuIgE was at 1:500. No significant differences were seen. Data from 3 independent assays were pooled and shown together (mean±SEM).

FIG. 7. Microarray technology. Comparison of IgE binding to 16mer Epitope 3-Core, the 20mer sequence and the mimotope. P was substituted with h in all peptides. IgE binding to the mimotope at 0.3 IU/ml was statistically different (p<0.05); one way ANOVA with multiple comparisons. Data from 3-4 independent assays were pooled and shown together (mean±SEM). *Only at Ara h 2 sIgE of 0.3 kU/L, the mimotope bound significantly more IgE than did Epitope 3-Core (p<0.05). There was no significant difference among any other comparisons.

FIG. 8. Dot blot with purified peptides. Eighteen 16mer peptides (95-97% pure) of Epitope 3-Core were spotted onto nitrocellulose, probed with the serum pool and developed for IgE binding. Peptide #1 was the native sequence. Each subsequent peptide had alanine substituted for sequential amino acids as shown in FIGS. 1 and 2. IgE binding to the peptides was not detectable whereas binding to a sample containing native Ara h 2 and Ara h 6 was easily seen.

### DETAILED DESCRIPTION

Disclosed herein are compositions and methods for predicting or assessing the presence and/or severity of IgE-mediated peanut allergy in a subject. The disclosed methods and compositions may comprise one or more peptides or mimotopes of Ara h 2, specifically Epitope 3, or a portion thereof. In many embodiments, the peptides and mimotopes may comprise at least one DPY_P sequence, in many cases the proline, “P,” in the sequence may be hydroxylated (i.e. “h”) and may be represented by h. In many embodiments, substituting a P->h within the disclosed peptide and mimotope sequences may increase binding of IgE from the subject. In many embodiments, enhanced binding of IgE from a subject indicates that the subject suffers from an IgE-mediated peanut allergy.

IgE binding to the 2S albumin allergens of peanuts (Ara h 2 and Ara h 6) correlates with the presence of clinically relevant IgE-mediated food allergy. IgE binding to specific linear epitopes of Ara h 2 can enhance the diagnostic accuracy of Ara h 2-specific IgE (Ara h 2-sIgE) and is associated with clinical outcomes such as the presence of clinically relevant peanut allergy, sensitivity to challenge and attainment of sustained unresponsiveness. Structural aspects of IgE binding to linear epitopes are also relevant, as linear sequences can fold together to form surface patches (conformational epitopes) for IgE binding and conformational epitopes are involved in overall IgE binding.

Optimal binding of IgE, from peanut allergic patients, to epitope 3, located in a central, disordered loop region of Ara h 2, is dependent upon post-translational hydroxylation of specific prolines in the native protein. In the absence of this specific hydroxylation, IgE binding is reduced 100-1000 fold. The discovery of IgE binding mimotopes of Ara h 2 that contain the core DPY_P motif suggests the involvement of conformational epitopes related to this region and this has been further supported by studies of Ara h 2/Ara h 6 chimeras.

Measuring IgE binding to the DPYSP motif of Ara h 2 enhances in vitro diagnostics. Applicants performed ELISAs and alanine scanning, using microarrays, to determine a core sequence of epitope 3 (Epitope 3-Core) and the relative importance of individual amino acids throughout its sequence and throughout the sequence of a mimotope containing the central DPY_P motif of epitope 3. The relevance of these studies was enhanced by changing the “P” of the DPYSP motif in epitope 3 and in the mimotope to “h” prior to alanine scanning. In many embodiments, the presence of hydroxylated proline instead of proline, within the amino acid sequence of the disclosed peptides or mimotopes, may result in an increase of IgE binding from serum of a subject suffering from or at risk of developing IgE-associated peanut allergy. In many embodiments, the increase may be more than about 10%, 20%, 30%, 40%, 50%, 55%, 60%, 65%, 70%, 75%, 80%, 85%, 90%, 95%, 100%150%, or 200%, and less than about 500%, 400%, 350%, 300%, 250%, 200%, 150%, 100%, 90%, 80%, 70%, 60%, 50%, or 40%, relative to binding to an unsubstituted peptide. In many embodiments, the substitution may be useful in enhancing the sensitivity of the disclosed methods. The precision of these data are enhanced by statistical analysis of combined data from both pooled and individual sera of individuals with strong clinical histories and a high likelihood of clinical allergy to peanut and/or tree nuts.

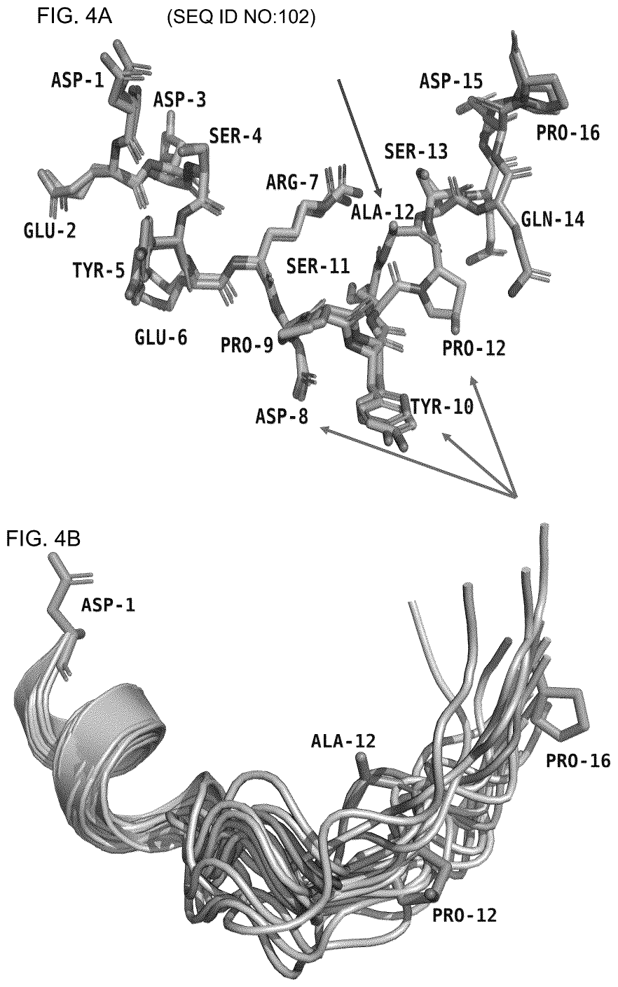

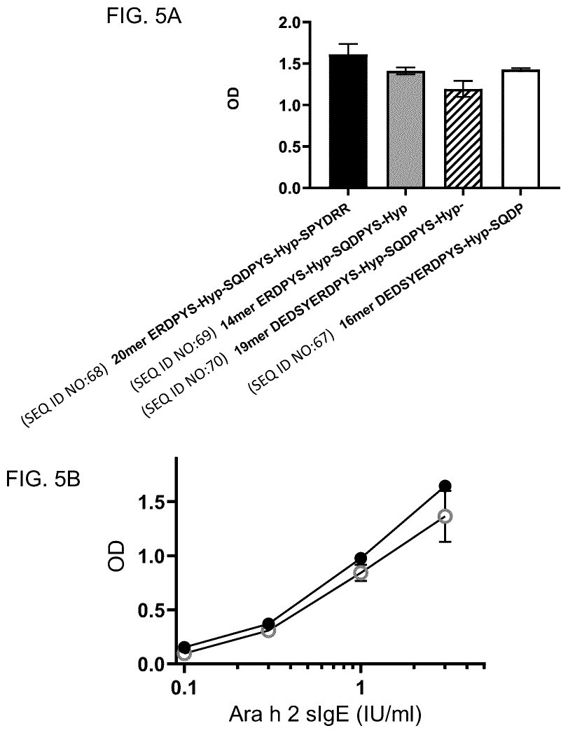

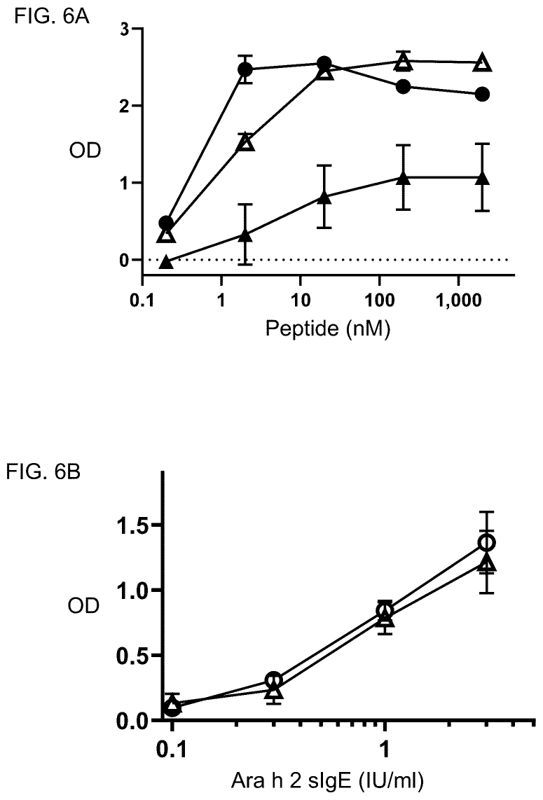

Prior to performing alanine scanning of the unstructured region of Ara h 2.01, a 20mer peptide of this region (ERDPYShSQDPYShSPYDRR; positions ww-xx of the Ara h 2 protein; SEQ ID NO:68) which contains two copies of the DPYSh (SEQ ID NO:71) domain and shortened versions were synthesized with the Biotide® protocol and probed with pooled sera. A core 16mer sequence, DEDSYERDPYShSQDP (positions yy-zz; SEQ ID NO:67), containing only one DPYSh (SEQ ID NO:71) sequence, here called Epitope 3-Core, was found to bind IgE almost as well as the 20mer sequence containing two copies of the DPYSh (SEQ ID NO:71) motifs (FIG. 5A). The presence of only one copy of the DPYSh (SEQ ID NO:71) motif allowed for efficient alanine screening. The strong binding of IgE to Epitope 3-Core compared to the 20mer peptide was verified using pure peptides (FIG. 5B). Sera were diluted to contain the same concentrations of Ara h 2-sIgE (usually 10 IU/ml) or at various concentrations for dose response assays and assayed in duplicate arrays on 3-4 separate days as previously described except for use of biotinylated anti-IgE (clone HP6029 from Southern Biotech at 1:1,000). A147, a 12mer mimotope with sequence LLDPYAPRAWTK (SEQ ID NO:73), contains the sequence DPY_P (SEQ ID NO:101), but did not bind IgE well. However, when the P of A147 was substituted with h (i.e. LLDPYAhRAWTK; SEQ ID NO:2), binding was dramatically enhanced (FIG. 6A) and the substituted mimotope bound amounts of IgE similar to that of the Epitope 3-Core peptide (FIG. 6B). In the microarray format, the mimotope tended to bind more IgE at lower doses of serum than either the 20mer sequence or Epitope 3-Core but at higher doses of serum this effect was lost (FIG. 7).

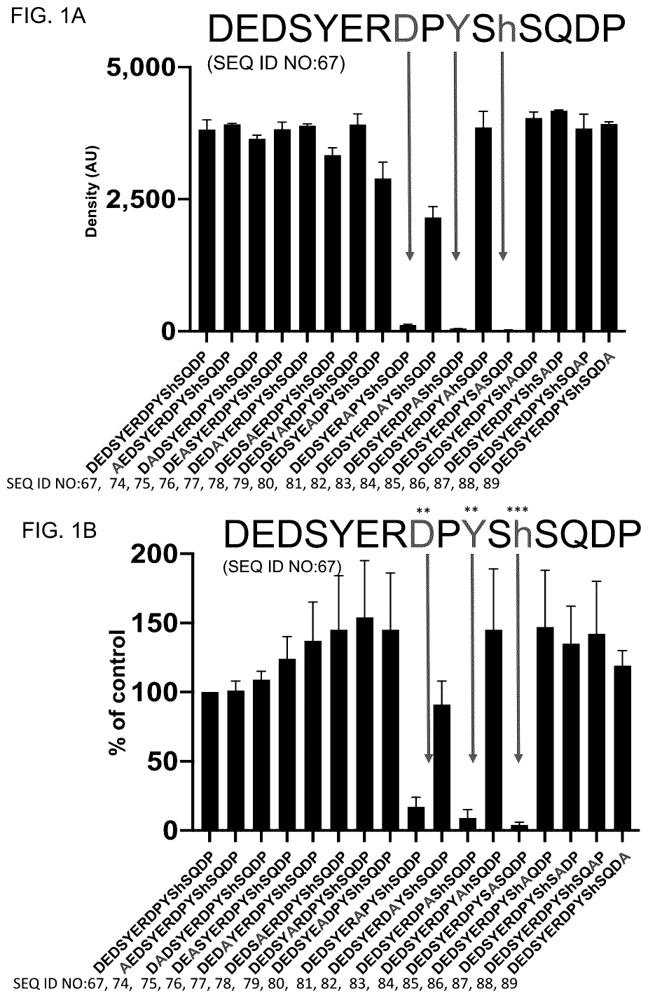

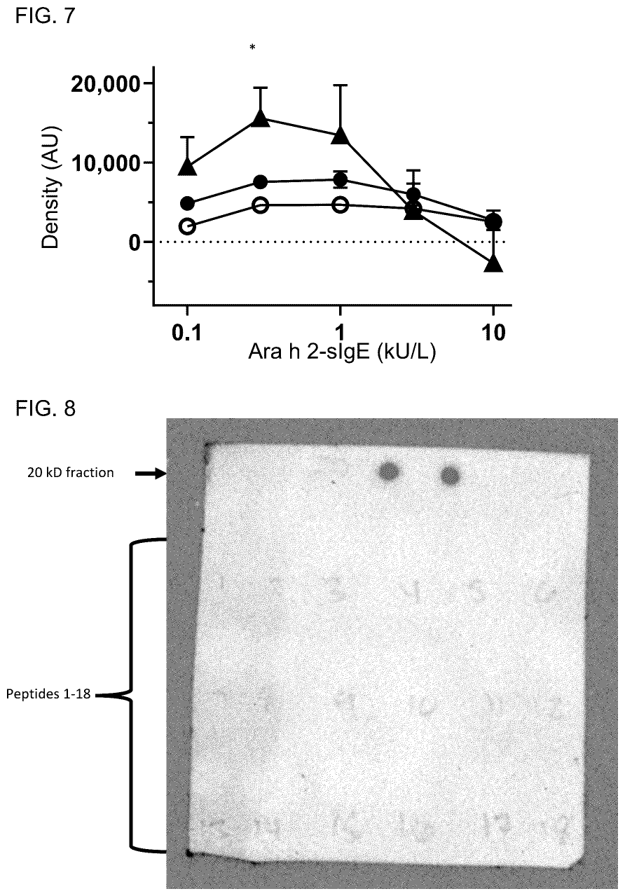

Direct spotting of peptides to nitrocellulose did not generate a perceptible signal for IgE binding (FIG. 8) whereas use of microarrays to analyze the disclosed peptides generated signals of 5-10 times background. FIG. 1 shows data comparing the binding of IgE from the serum pool to alanine substituted peptides from one individual assay (FIG. 1A) and for summary data from 4 separate assays (FIG. 1B). Of the 16 amino acids present in Epitope 3-Core, independently changing 3 led to significant diminution of IgE binding. These are amino acids D8 (17±7%; P<0.01), Y10 (9±6%; P<0.01) and hyp12 (hydroxyproline 12) (4±2%; P<0.001) (FIG. 1B). In many embodiments, substitution of one or more of the aspartic acid, tyrosine, or proline/hydroxylated proline within the D-P-Y-S-P/h sequence of a peptide or mimotope, for example substituting for an alanine or “A,” may result in a reduction of IgE binding from serum of a subject suffering from or at risk of developing IgE-associated peanut allergy. In many embodiments, the reduction in IgE binding may be more than about 50%, 55%, 60%, 65%, 70%, 75%, 80%, 85%, 90% or 95% relative to binding to an unsubstituted peptide. In many embodiments, the substitution may be various non-native residues, such as an alanine residue.

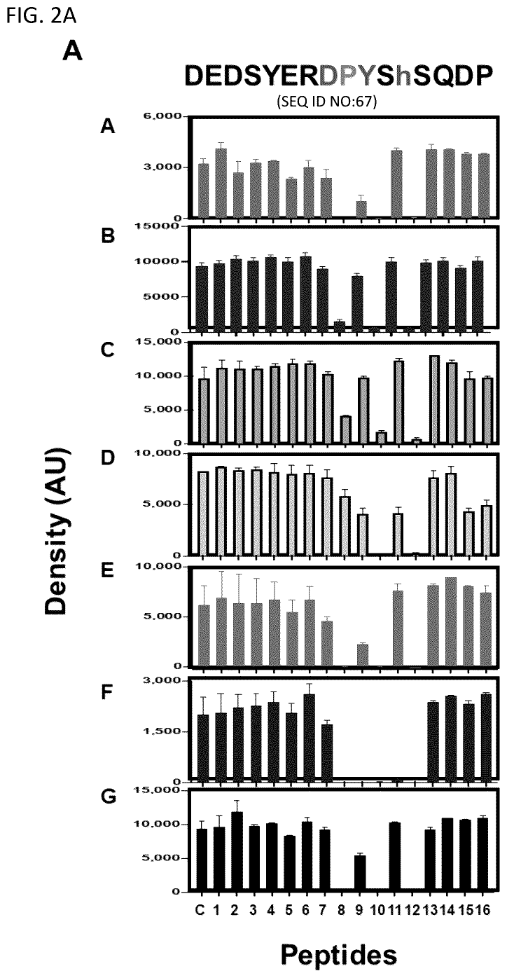

Results with individual sera revealed more nuance. FIG. 2 shows data for 7 sera with data from one individual assay (FIG. 2A) and summary data from 4 separate assays (FIG. 2B). Table 1 shows the statistical significance for alanine substitution for each amino acid. While D8, Y10, and h12 were relevant for most sera (FIG. 2B), results for the other positions were more variable. For example, amino acid D3 was important for serum D218, amino acid P16 for D213, amino acid P9 for sera D213, D216, D217 and D218 (P<0.05 for each) and amino acid D15 is important for binding of IgE from sera D107 and D213 (P<0.01). There were no immediately apparent clinical features (Table 2) that corresponded to these findings.

TABLE 1
Statistical significance of alanine scans for Epitope 3-Core (FIGS. 1B and 2B)

| | (SEQ ID NO: 67) Amino acid altered to Alanine | | | | | | | | | | | | | | | |
|---|---|---|---|---|---|---|---|---|---|---|---|---|---|---|---|---|
| Sera | D | E | D | S | Y | E | R | D | P | Y | S | h | S | Q | D | P |
| Serum | | | | | | | | ** | | ** | | *** | | | | |
| Pool | | | | | | | | | | | | | | | | |
| D107 | | | | | | | | *** | | *** | | *** | | | ** | |
| D197 | | | | | | | | **** | | **** | | *** | | | | |
| D199 | | | | | | | | * | | ** | | *** | | | | |
| D213 | | | | | | | | | * | **** | * | **** | | | ** | * |
| D216 | | | | | | | | *** | * | *** | | *** | | | | |
| D217 | | | | | | | | *** | * | *** | | *** | | | | |
| D218 | | | * | | | | | *** | * | **** | | **** | | | | |

P < 0.05;
** P < 0.01;
*** P < 0.001;
**** P < 0.0001

TABLE 2
Clinical and Laboratory Data

| | | | | | | Ara h |
|---|---|---|---|---|---|---|
| Subject | | | | Clinical | PN-sIgE | 2-sIgE |
| Serum # | Age | Gender | Race** | Reaction*** | (kU/L) | (kU/L) |
| D19* | 29 | M | C | S, R | 59 | 17 |
| D60* | 16 | M | C | S, AE, R | 159 | 24 |
| D64* | 9 | F | C | S, AE, R | 848 | 571 |
| D70* | 13 | M | C | S, AE, R | 500 | 161 |
| D77* | 11 | M | C | S, R | 48.7 | 50 |
| D80* | 13 | M | C | GI | 164 | 78 |
| D107^{†} | 32 | F | H | S, AE, GI, R | 95.5 | 64 |
| D197^{†} | 8 | F | C | GI, N | 230 | 166 |
| D199^{†} | 11 | F | C | S, AE, GI | 449 | 221 |
| D213^{†}* | 6 | M | C | AE, R | 609 | 455 |
| D216^{†}* | 7 | F | C | S, AE, GI | 141 | 73 |
| D217^{†} | 7 | M | C | S, R | 285 | 231 |
| D218^{†}* | 15 | F | C | S, AE, R | 318 | 255 |
| D224* | 12 | M | C | S, AE, GI | 144 | 144 |

^{†}Studied individually
*Present in the serum pool.
**Race or ethnicity: C: Caucasian, H: Hispanic
***Clinical reactions: S: Skin erythema or hives, R: wheezing and/or respiratory distress, A: Anaphylaxis, AE: Angioedema, GI: Gastrointestinal symptoms, N: nasal symptoms of rhinorrhea and/or congestion.

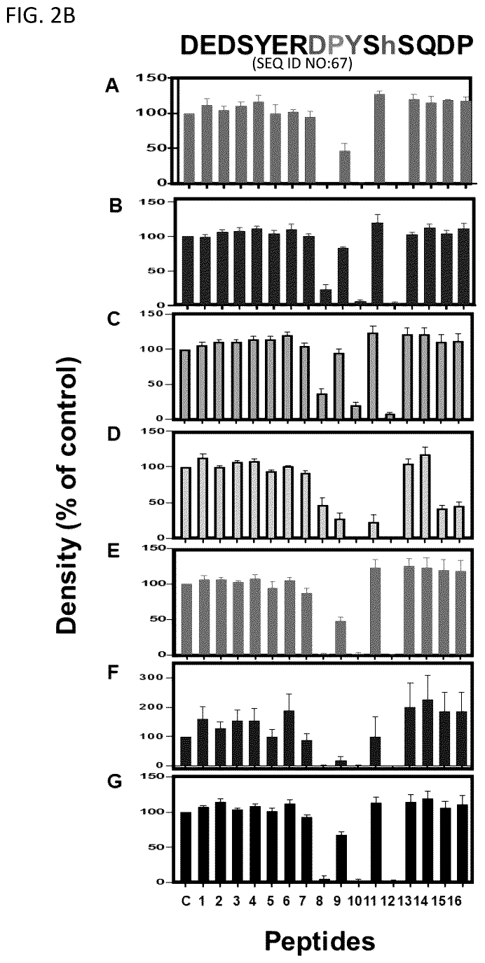

IgE binding to the mimotope, A147 (FIG. 3), was similar to that seen for binding to epitope 3 discussed above. Specifically, amino acids Y5 (19±5%; P=0.02) and hyp7 (−8±4%; P=0.001) being relevant for robust IgE binding. However, in contrast to the finding with Epitope 3-Core, the D3 in the DPY_P (SEQ ID NO:101) motif was not relevant.

Modeling the exact effect of each residue on the overall structure of the epitope 3 region is difficult for two reasons. The first is that the region is very flexible and the X-ray crystal structure of Ara h 2 lacks many of the residues in this area. The second difficulty is the need to account for the post translational addition of the hydroxyl group on the proline, which is not present in any experimental structure done with recombinant 2S-albumins. We used AlphaFold 2 to suggest conformations for this loop that could form as the predictive accuracy of AlphaFold models comes close to the accuracy of experimental methods. Given limitations in the available templates, all models were generated using P, not the h.

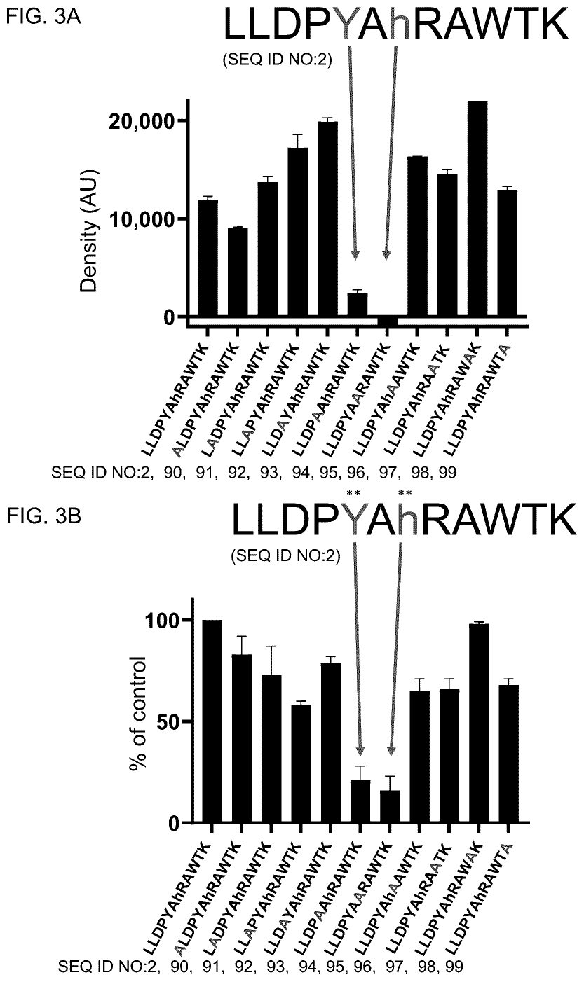

Applicants generated a 3D model of this area of Ara h 2, followed by another 16 models with each residue individually replaced by Ala. The best ranked 3D model of Ara h 2 shows a large surface accessible loop consisting of the C-terminal amino acids of Epitope 3-Core, residue positions D8 to P16, with the P12 residue on the outer surface of the fold on the same side as the other residues (D8 and Y10) found to relevant for specific IgE recognition (FIG. 4A, blue arrows). Hydroxylation of P12 to h12 is expected to enhance this residue's hydrophilicity and surface propensity, and may enhance the stability of the trans conformation.

Although the other alanine substitutions made little difference to the overall backbone fold of the peptide (FIG. 4B), changing P12 to A dramatically changed the side chain orientation: A12 points toward the opposite side of the structured region (orange in both FIG.s). As the position of the hydroxyl group in the WT Ara h 2 model is highly solvent exposed, Applicants hypothesize that the hydroxyl group forms a hydrogen bond with IgE. While amino acid mutations in the first 7 peptide residues had little effect, they caused increasing perturbation in the C-terminal residues This could account for why substitution at residues D8 to P16 also affect IgE binding for some sera.

The epitope of Ara h 2, referred to as epitope 3 in this report, may be important to understanding why Ara h 2 is such a potent allergen and likely contributes to conformational epitopes. It is also a unique feature of this 2S-albumin compared to other known allergens in this PFAM. This epitope is highly surface exposed, located within a structurally disordered region of Ara h 2 (FIG. 4) that contains duplications of a core DPYSh (SEQ ID NO:71) motif. The flexibility of this region may account for why 3 amino acids, D, Y and h, are independently involved in IgE binding to the 16mer, Epitope 3-Core, but only the Y and h are comparably involved in a related 12mer mimotope that contains the core DPY_h sequence. Of interest, non-core amino acids (e.g. P9 and D15) contributed to IgE binding to the Epitope 3-Core for selected sera (Table 1) but not for the 12mer mimotope (data not shown).

IgE is approximately 10⁵ less prevalent in serum or plasma than is IgG and its binding is therefore more difficult to measure. This is reflected in the literature where one can find reports of IgE binding to intact proteins bound to nitrocellulose but not to peptides unless the peptides were synthesized directly onto the membrane. We recapitulated this finding in that IgE binding to peptides could not be detected following blotting of the peptides onto nitrocellulose (FIG. 8) but could easily be detected when the peptides were assayed in a biotin-based ELISA (FIGS. 5A-6B) or printed onto microarrays (FIG. 7 and FIGS. 1-3).

Applicants used a statistical analysis of the large body of data generated from our peptide microarrays rather than using an arbitrary % binding as a way of assessing the significance of specific amino acids for binding to polyclonal IgE in sera. In addition, Applicants have applied molecular modeling to identify structural changes induced by alanine substitution. Finally, many studies of clinical specimens do not include hydroxyproline in the peptide sequences, a modification that is involved in optimal IgE binding. In addition, the alanine scanning analysis and modeling only examined the individual contribution of individual amino acids—Applicants believe that interactions with neighboring amino acids may also contribute to forming conformational epitopes.

In conclusion, these results further emphasize the relevance of the DPYSh (SEQ ID NO:71) motif of epitope 3 and the DYP_h motif of a related mimotope for IgE binding while suggesting that certain conformations of this flexible area may also contribute to its immunogenicity. These results may aid in the design of hypoallergenic forms of Ara h 2.

The following terms and phrases include the meanings provided below. The provided definitions are intended to aid in describing particular embodiments, and are not intended to limit the claimed compositions, methods, compounds, systems, and therapies. Unless otherwise defined, all technical and scientific terms used herein have the same meaning as commonly understood by one of ordinary skill in the art to which this disclosure belongs. If there is an apparent discrepancy between the usage of a term in the art and its definition provided herein, the definition provided within the specification shall prevail.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Amino acid identity,” “residue identity,” “identity,” and the like, as used herein refers to the structure of the functional group (R group) on the poly peptide backbone at a given position. Naturally occurring amino acid identities are (name/3-letter code/one-letter code): alanine/ala/A; arginine/arg/R; asparagine/asn/N; aspartic acid/asp/D; cysteine/cys/C; glutamine/gln/Q; glutamic acid/glu/E; glycine/gly/G; histidine/his/H; isoleucine/ile/I; leucine/leu/L; lysine/lys/K; methionine/met/M; phenylalanine/phe/F; proline/pro/P; serine/ser/S; threonine/thr/T; tryptophan/trp/W; tyrosine/tyr/Y; and valine/val/V.

An amino acid within a molecule may be substituted to create an engineered molecule. The amino acid (aa or a.a.) residue can be replaced by a residue having similar physiochemical characteristics, that is a ‘conservative substitution’—e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, for example based on size, charge, polarity, hydrophobicity, chain rigidity/orientation, etc., are well known in the art of protein engineering. Polypeptides comprising conservative amino acid substitutions can be tested in any one of the assays described herein to confirm that a desired activity, e.g. binding, specificity, and/or function of a native or reference polypeptide is achieved.

While conservative substitutions within a protein, i.e. buried or non-solvent accessible residues/positions, may in some cases alter the structure of the protein or affect folding of the protein, conservative substitutions at or near the protein's surface, i.e. exposed or solvent accessible residues/positions may cause little or no discernable change to the protein's structure and/or function, unless the altered surface protein is necessary for an interaction with another molecule, peptide, or protein. It is well within the abilities of the skilled artisan to alter the disclosed protein sequences by introducing conservative substitutions at up to 20% of the residues/positions without disrupting or changing the protein's structure and/or function.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: leucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

“Diagnose” or “diagnostic” refers identifying the presence or absence of or nature of a disease or disorder. Such detection methods can be used, for example, for early diagnosis of the condition, to determine whether a subject is predisposed to a disease or disorder, to monitor the progress of the disease or disorder or the progress of treatment protocols, to assess the severity of the disease or disorder, to forecast the an outcome of a disease or disorder and/or prospects of recovery, or to aid in the determination of a suitable treatment for a subject.

A “patient” or “subject” includes a mammal or animal, such as a human, cow, horse, sheep, lamb, pig, chicken, turkey, quail, cat, dog, mouse, rat, rabbit, or guinea pig. The animal can be a mammal such as a non-primate or a primate (e.g., monkey and human). In one embodiment, a patient is a human, such as a human infant, child, adolescent, or adult of any or indeterminant sex.

“Prevention” as used herein means the avoidance of the occurrence or of the re-occurrence of a disease, disorder, or condition as specified herein, by the administration of a composition, compound, treatment, or therapy according to the present disclosure to a subject in need thereof.

“Subject in need,” “patient” or those “in need of treatment” include those already with existing disease (i.e. allergies, for example, without limitation, IgE-associated peanut allergy), as well as those at risk of or susceptible to the disease. The terms also include human and other mammalian subjects that receive either prophylactic or therapeutic treatments as disclosed herein.

The terms “treat,” “treating,” and “treatment” refer to eliminating, reducing, suppressing, or ameliorating, either temporarily or permanently, either partially or completely, a clinical symptom, manifestation or progression of an event, disease or condition associated with immune disorders and diseases described herein. As is recognized in the pertinent field, methods and compositions employed as therapies may reduce the severity of a given disease state but need not abolish every manifestation of the disease to be regarded as useful. Similarly, a prophylactically administered treatment need not be completely effective in preventing the onset of a condition to constitute a viable prophylactic method or agent. Simply reducing the impact of a disease (for example, as disclosed herein, IgE-associated peanut allergy, etc. and/or reducing the number or severity of associated symptoms, or by increasing the effectiveness of another treatment, or by producing another beneficial effect), or reducing the likelihood that the disease will occur or worsen in a subject, is sufficient. One embodiment of the present disclosure is directed to a method for determining the efficacy of treatment comprising administering to a patient therapeutic treatment in an amount, duration, and repetition sufficient to induce a sustained improvement over pre-existing conditions, or a baseline indicator that reflects the severity of the particular disorder.

### EXAMPLES

### Example 1—Methods

### Sera and Serum Pool

Sera were obtained from 14 peanut-allergic subjects with physician-diagnosed peanut allergy each of whom had a peanut-specific IgE ≥15 kU/L and Ara h 2-specific IgE >5 kU/L (ImmunoCap, Phadia; Uppsala, Sweden) making it highly likely that each would fail a blinded peanut challenge. To produce the serum pool, 10 individual sera were pooled with the concentration of Ara h 2-sIgE normalized so that no one serum was predominant (Table 2). Seven sera were studied in detail, 3 of these were included in the serum pool and 4 independent of the serum pool (Table 2). The Colorado Multiple Institutional Review Board (COMIRB) approved this study. All adult patients and the parents or guardians of minors gave written informed consent. Minors who were >6 years of age, signed an assent.

### Peptides

Peptides for ELISAs were synthesized with an N-terminal linker arm (Ttds; JPT Peptides, Berlin) followed by biotin at the N terminus. Prior to generating microarrays with high fidelity sequences, we performed screening assays using the Biotide approach (JPT Peptides, Berlin) in which multiple related peptides are synthesized in a cost-effective manner at approximately 20% purity. For validation, selected peptides were also synthesized by standard methods and purified (95-97% pure) by HPLC (JPT Laboratories, Berlin). For all peptides of epitope 3 and the related mimotope studied here, trans-4-hydroxyproline (h) was substituted for second proline in the DPY_P motif as this substitution may enhance binding of IgE. Peptides for microarray assays were synthesized with the Ttds linker arm on the N terminus and printed onto microscope slides treated with a proprietary 3-dimensional surface by JPT Peptides (Berlin).

### ELISAs

Streptavidin (InVitrogen) (5 μg/ml) was bound to micro titer plates by overnight incubation in PBS, the plates were washed once with PBST (0.05% Tween 20) and 50 μL of peptides were added at a final concentration of 1-5 nM in PBST for 1 hour at room temperature. Unoccupied streptavidin sites were then saturated with 400 μM biotin in PBS and the samples were probed with diluted sera from allergic subjects diluted in PBST with 0.1% albumin to contain 10 IU/ml of Ara h 2-sIgE. After overnight incubation at 4° C., slides were washed, incubated with HRP-labeled murine monoclonal anti-human IgE (clone HP6029; Southern Biotech 1:500-1:3,000), washed again and developed with TMB (ThermoFisher Scientific). Background binding of serum without peptide (OD=approximately 0.100) was subtracted. We also examined the background of peptide without serum and this was similar to, or lower than, the background of serum without peptide.

### Microarrays

Each microscope slide contained 21 microarrays and within each microarray, peptides were printed in triplicate. 10-15 spots were not printed with any peptide as control spots. Sera were diluted to contain the same concentrations of Ara h 2-sIgE (usually 10 IU/ml) or at various concentrations for dose response assays and assayed in duplicate arrays on 3-4 separate days as previously described except for use of biotinylated anti-IgE (clone HP6029 from Southern Biotech at 1:1,000). For each microarray assay, the density units of three observations within each microarray were averaged and then the data from duplicate microarrays were averaged. To combine data from multiple independent assays, the signal within each assay was normalized to that of a wild-type peptide and the signal was expressed as a percent of that with the original peptide. Background binding of serum without peptide (300-500 AU) was subtracted. We also examined the background of peptide without serum. Only for the mimotope, this background was higher than the background of serum without peptide and these values (5,000-10,000 AU), which were quite high, were subtracted from the signal of serum with peptide (which bound IgE very strongly (20,000-30,000 AU), instead of subtracting the background of serum without peptide.

### Alanine Scans

Linear sequences of epitope 3 and the mimotope as well as derivative peptides in which each amino acid was sequentially changed to alanine were studied. Within each peptide, amino acids that, when replaced with alanine, showed significant diminution of binding were considered to be critical amino acids.

### Dot Blots

Peptides were synthesized without the above-referenced N-terminal linker or biotin. Peptides were purified to >95% purity by JPT peptides (Berlin). 0.25 μg (124 pmoles) of each peptide was spotted onto nitrocellulose. One μg (66 pmoles) of a 20 kD fraction of crude peanut extract containing a combination of Ara h 2 and Ara h 6 was spotted as a positive control. The blot was blocked with 5% milk protein, probed with diluted serum pool (containing 16 kU/ml of Ara h 2 sIgE) and then developed first with unlabeled mouse anti-human IgE (clone HP6029 from Southern Biotech; 1:500) followed by biotinylated goat-anti mouse Fc Gamma (Jackson Immuno Research; 1:1,000) and finally with HRP-streptavidin (InVitrogen; 1:3,0000). The blot was then developed with TMP.

### Statistical Analysis

GraphPad Prism (versions 8.4-9.5 for the Macintosh (GraphPad; La Jolla, CA)) was used to generate graphs and for statistical analysis. The following analyses were performed: 1) for dose response curves, multiple unpaired t test with Welch correction for unequal variances; 2) to combine data from multiple assays, the data were normalized within each assay based on IgE binding to the native sequence (epitope 3) or the original sequence (mimotope A147) and expressed binding as percent of binding and 3) to compare differences in IgE binding for a specific alanine substitution, one way ANOVA followed by Dunnet's multiple comparison test.

### Example 2—Results

Prior to performing alanine scanning of the unstructured region of Ara h 2.01, a 20mer peptide of this region (ERDPYShSQDPYShSPYDRR; (SEQ ID NO:68)) which contains two copies of the DPYSh domain and shortened versions were synthesized with the Biotide® protocol and probed with pooled sera. A core 16mer sequence, DEDSYERDPYShSQDP (SEQ ID NO:67), containing only one DPYSh sequence, here called Epitope 3-Core, was found to bind IgE almost as well as the 20mer sequence containing two copies of the DPYSh (SEQ ID NO:71) motifs (FIG. 5A). The presence of only one copy of the DPYSh (SEQ ID NO:71) motif allowed for efficient alanine screening. The strong binding of IgE to Epitope 3-Core compared to the 20mer peptide was verified using pure peptides (FIG. 5B). Sera were diluted to contain the same concentrations of Ara h 2-sIgE (usually 10 IU/ml) or at various concentrations for dose response assays and assayed in duplicate arrays on 3-4 separate days as previously described except for use of biotinylated anti-IgE (clone HP6029 from Southern Biotech at 1:1,000). The 12mer mimotope, A147 (LLDPYAPRAWTK; SEQ ID NO:73), which contains the sequence DPY_P, did not bind IgE well but, when the P was substituted with h (LLDPYAhRAWTK; SEQ ID NO:2), binding was dramatically enhanced (FIG. 6A) and bound similar amounts of IgE as did Epitope 3-Core (FIG. 6B). In the microarray format, the mimotope tended to bind more IgE at lower doses of serum than either the 20mer sequence or Epitope 3-Core but at higher doses of serum this effect was lost (FIG. 7).

Direct spotting of peptides to nitrocellulose did not generate a perceptible signal for IgE binding (FIG. 8) whereas the microarray approach generated signals of 5-10 times background. FIG. 1 shows data comparing the binding of IgE from the serum pool to the alanine substituted peptides from one individual assay (FIG. 1A) and for summary data from 4 separate assays (FIG. 1B). Of the 16 amino acids present in Epitope 3-Core, independently changing 3 lead to significant diminution of IgE binding. These are amino acids D8 (17±7%; P<0.01), Y10 (9±6%; P<0.01) and hyp12 (hydroxyproline 12) (4±2%; P<0.001) (FIG. 1B).

Results with individual sera revealed more nuance. FIG. 2 shows data for 7 sera with data from one individual assay (FIG. 2A) and summary data from 4 separate assays (FIG. 2B). Table 1 shows the statistical significance for alanine substitution for each amino acid. While D8, Y10, and h12 were relevant for most sera (FIG. 2B), results for the other positions were more variable. For example, amino acid D3 was important for serum D218, amino acid P16 for D213, amino acid P9 for sera D213, D216, D217 and D218 (P<0.05 for each) and amino acid D15 is important for binding of IgE from sera D107 and D213 (P<0.01). There were no immediately apparent clinical features (Table 2) that corresponded to these findings.

IgE binding to the mimotope, A147 (FIG. 3), was similar to that seen for epitope 3, with the amino acids Y5 (19±5%; P=0.02) and hyp7 (−8±4%; P=0.001) being relevant for robust IgE binding. However, in contrast to the finding with Epitope 3-Core, the D3 in the DPY_P motif was not relevant.

Modeling the exact effect of each residue on the overall structure of the epitope 3 region is difficult for two reasons. The first is that the region is very flexible and the X-ray crystal structure of Ara h 2 lacks many of the residues in this area. The second difficulty is the need to account for the post translational addition of the hydroxyl group on the proline, which is not present in any experimental structure done with recombinant 2S-albumins. We used AlphaFold 2 to suggest conformations for this loop that could form as the predictive accuracy of AlphaFold models comes close to the accuracy of experimental methods. Given limitations in the available templates, all models were generated using P, not the h.

Applicants first generated a 3D model of this area of Ara h 2, followed by another 16 models with each residue individually replaced by Ala. The best ranked 3D model of Ara h 2 shows a large surface accessible loop consisting of the C-terminal amino acids of Epitope 3-Core, residue positions D8 to P16, with the P12 residue on the outer surface of the fold on the same side as the other residues (D8 and Y10) found to relevant for specific IgE recognition (FIG. 4A, blue arrows). Hydroxylation of P12 to h12 is expected to enhance this residue's hydrophilicity and surface propensity, and may enhance the stability of the trans conformation.

Although the other alanine substitutions made little difference to the overall backbone fold of the peptide (FIG. 4B), changing P12 to A dramatically changed the side chain orientation: A12 points toward the opposite side of the structured region (orange in both figures). As the position of the hydroxyl group in the WT Ara h 2 model is highly solvent exposed, it was hypothesized that the hydroxyl group may form a hydrogen bond with IgE. While amino acid mutations in the first 7 peptide residues had little effect, they caused increasing perturbation in the C-terminal residues This could account for why substitution at residues D8 to P16 also affect IgE binding for some sera.

TABLE 3
Sequences disclosed

| SEQ ID. NO. | NAME | SEQUENCE |
|---|---|---|
| 1 | Ara_h_2-Mimotope_A05 | MDRHAQNYAERS |
| 2 | Ara_h_2-Mimotope_A147 P → h | LLDPYAhRAWTK |
| 3 | Ara_h_2-Mimotope_A19 P → h | QDGSSDPYAhAA |
| 4 | Ara_h_2-Mimotope_A24 P → h | DRDPFAhSFGAW |
| 5 | Ara_h_2-Mimotope_A39 P → h | HYDPYAhSISTV |
| 6 | Ara_h_2-Mimotope_A42 P → h | ASPRDPYMhSPT |
| 7 | Ara_h_2-Mimotope_A45 P → h | DPYGGhKFHLRV |
| 8 | Ara_h_2-Mimotope_A52 P → h | SHDPYAhSDSSV |
| 9 | Ara_h_2-Mimotope_A68 P → h | YLPWDPYGGhQR |
| 10 | Ara_h_2-Mimotope_A74 P → h | TPPTIRDPYAhL |
| 11 | Ara_h_2_Mimotope_A92-A | ALNLTSWPSRYA |
| 12 | Ara_h_2-Mimotope_A93 P → h | VERDPYAhAPEY |
| 13 | Ara_h_2-Mimotope_A104 P → h | EDVTQDPFAhTR |
| 14 | Ara_h_2-Mimotope_A119 P → h | ASHTLMADPYAh |
| 15 | Ara_h_2-Mimotope_A126 P → h | ANRNVYADPVKh |
| 16 | Ara_h_2-Mimotope_A147 | LLDPYAPRAWTK |
| 17 | Ara_h_2-Mimotope_A150 P → h | THQDPYAhRLGS |
| 18 | Ara_h_2-Mimotope_A167 P → h | SNWDPFAhRAAG |
| 19 | Ara_h_2-Mimotope_A171 P → h | EFPTLTRDPYRh |
| 20 | Ara_h_2-Mimotope_A174 P → h | APYWPGADPYRh |
| 21 | Ara_h_2-Mimotope_A194 P → h | HFHTVDPFAhSG |
| 22 | Ara_h_2-Mimotope_A212 P → h | GPDPYAhSMISN |
| 23 | Ara_h_2-Mimotope_A239 P → h | GGSSGDPYAhSH |
| 24 | Ara_h_2-Mimotope_A353 P → h | ASTYSSPDPYRh |
| 25 | Ara_h_2-Mimotope_A371 P → h | VNAHDPFAhSLP |
| 26 | Ara_h_2-Mimotope_M12 | SQDSSVTPYLLE |
| 27 | Ara_h_2-Mimotope_M19 | TIVGRQNSTIAA |
| 28 | Ara_h_2-Mimotope_M34 | SDIGRONFLQAL |
| 29 | Ara_h_2-Mimotope_M47 | SHYGVEVDLMSI |
| 30 | Ara_h_2-Mimotope_M66 | MLEEQCQSRLSC |
| 31 | Ara_h_2-Mimotope_M67 | DRQNEEYMRLRV |
| 32 | Ara_h_2-Mimotope_M68 | WDPPGRONLOHR |
| 33 | Ara_h_2-Mimotope_M79 | PMQPGGQTRGLP |
| 34 | Ara_h_2-Mimotope_M82 | ADCCRYIAIPGS |
| 35 | Ara_h_2-Mimotope_M107 | GAYPSFDERGLS |
| 36 | Ara_h_2-Mimotope_M113 | VSNHGGRQNSFL |
| 37 | Ara_h_2-Mimotope_M117 | DLIVGRQNTTSY |
| 38 | Ara_h_2-Mimotope_M121 | ETKIVSLQELWI |
| 39 | Ara_h_2-Mimotope_M130 | MGDTEQWGTGTH |
| 40 | Ara_h_2-Mimotope_M131 | ASTCVRYQCAPL |
| 41 | Ara_h_2-Mimotope_M132 | NPVTSSVWFHP |
| 42 | Ara_h_2-Mimotope_M171 | NLEDRVOMRTLW |
| 43 | Ara_h_2-Mimotope_M253 | QFGCEARACNLP |
| 44 | Ara_h_2-Mimotope_F101 | DYWPSFDLSWED |
| 45 | Ara_h_2-Mimotope_F107 | WGNWGLPGSTYA |
| 46 | Ara_h_2-Mimotope_F263 | NSFDALHSVGNH |
| 47 | Ara_h_2-MimotopeA248_Peptide_002 | CVPTRDPYAhRM |
| 48 | Ara_h_2-MimotopeA248_Peptide_021 | GCPTRDPYAhRM |
| 49 | Ara_h_2-MimotopeA248_Peptide_040 | GVCTRDPYAhRM |
| 50 | Ara_h_2-MimotopeA248_Peptide_059 | GVPCRDPYAhRM |
| 51 | Ara_h_2-MimotopeA248_Peptide_066 | GVPKRDPYAhRM |
| 52 | Ara_h_2-MimotopeA248_Peptide_074 | GVPVRDPYAhRM |
| 53 | Ara_h_2-MimotopeA248_Peptide_077 | GVPTADPYAhRM |
| 54 | Ara_h_2-MimotopeA248_Peptide_078 | GVPTCDPYAhRM |
| 55 | Ara_h_2-MimotopeA248_Peptide_079 | GVPTDDPYAhRM |
| 56 | Ara_h_2-MimotopeA248_Peptide_080 | GVPTEDPYAhRM |
| 57 | Ara_h_2-MimotopeA248_Peptide_081 | GVPTFDPYAhRM |
| 58 | Ara_h_2-MimotopeA248_Peptide_084 | GVPTIDPYAhRM |
| 59 | Ara_h_2-MimotopeA248_Peptide_097 | GVPTRDCYAhRM |
| 60 | Ara_h_2-MimotopeA248_Peptide_115 | GVPTRDPYChRM |
| 61 | Ara_h_2-MimotopeA248_Peptide_131 | GVPTRDPYVhRM |
| 62 | Ara_h_2-MimotopeA248_Peptide_133 | GVPTRDPYYhRM |
| 63 | Ara_h_2-MimotopeA248_Peptide_140 | GVPTRDPYAhHM |
| 64 | Ara_h_2-MimotopeA248_Peptide_148 | GVPTRDPYAhSM |
| 65 | Ara_h_2-MimotopeA248_Peptide_164 | GVPTRDPYAhRP |
| 66 | Ara_h_2-MimotopeA248_Peptide_180 | GVPTRDPyAhRM |
| 67 | Ara_h_2 | DEDSYERDPYShSQDP |
| 69 | 20 mer peptide of FIG. 5A | ERDPYShSQDPYShSPYDRR |
| 70 | 14 mer peptide of FIG. 5A | ERDPYShSQDPYSh |
| 71 | 19 mer peptide of FIG. 5A | DEDSYERDPYShSQDPYSh |
| 72 | 16 mer peptide of FIG. 5A | DEDSYERDPYShSQDP |

Hyp/h = hydroxyproline
y = Tyr (Me) = 4-Methoxy-phenyalanine

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

All references disclosed herein, whether patent or non-patent, are hereby incorporated by reference as if each was included at its citation, in its entirety. In case of conflict between reference and specification, the present specification, including definitions, will control.

Although the present disclosure has been described with a certain degree of particularity, it is understood the disclosure has been made by way of example, and changes in detail or structure may be made without departing from the spirit of the disclosure as defined in the appended claims.

We claim:

1. A peptide having a sequence selected from SEQ ID NO: 1-66, wherein the peptide forms a linear mimotope of a conformational epitope of a 2S albumin of a peanut.

2. The peptide of claim 1, wherein the peptide has amino acid sequence SEQ ID NO: 2.

3. The peptide of claim 1, wherein the peptide binds serum IgE, has a sequence selected from SEQ ID NO: 2-10, 12-25, and 47-66, and wherein substitution of hydroxyproline for proline increases the amount of IgE bound by the peptide.

4. The peptide of claim 1, wherein the peptide has amino acid sequence SEQ ID NO: 11, the mimotope is of a conformation of an epitope of a peanut Ara h 2, and wherein the peptide binds more serum IgE than a peptide derived from wild-type Ara h 2.

5. A method of diagnosing a peanut allergy in a subject, the method comprising:
contacting serum from the subject with one or more peptides having an amino acid sequence selected from SEQ ID NO: 1-66; and
measuring the amount of serum IgE bound to the one or more peptides to generate a subject binding level;
comparing the subject binding level to
a first control binding level for an allergic patient, and
a second control binding level for a non-allergic patient; and
diagnosing the subject as having a peanut allergy if the subject binding level is equal or greater than the first control binding level.

6. The method of claim 5, wherein the one or more peptides has amino acid sequence of SEQ ID NO: 2.

7. The method of claim 5, wherein the contacting or measuring steps involve an immunoassay.

8. The method of claim 5, wherein the contacting or measuring steps involve a microarray.

9. The method of claim 5, wherein the subject biding level is greater than a binding a binding level measured using a wild-type peptide sequence.

10. The method of claim 6 further comprising diagnosing the subject as having an increased sensitivity to peanut allergens wherein subject binding level is statistically significantly greater than the first control binding level.

11. The method of claim 9, wherein the one or more peptides has the sequence of SEQ ID NO: 2.

12. A method of affecting IgE binding in a subject, the method comprising:
contacting serum from the subject with a peptide having the sequence of SEQ ID NO: 2, and
allowing the peptide to interact with IgE in the serum, wherein the peptide interferes with binding of IgE to at least one peptide having a native sequence comprising a DPYSP motif or a DPYSh motif.

13. The method of claim 12, wherein the peptide at least partially inhibits IgE binding to an Ara h 2 molecule.


SEQUENCE LISTING (102 sequences)

SEQ ID NO: 1 - protein, 12 aa, synthetic construct
MDRHAQNYAERS

SEQ ID NO: 2 - protein, 12 aa, synthetic construct
  MOD_RES 7: note = 4Hyp
LLDPYAXRAWTK

SEQ ID NO: 3 - protein, 12 aa, synthetic construct
  MOD_RES 10: note = 4Hyp
QDGSSDPYAXAA

SEQ ID NO: 4 - protein, 12 aa, synthetic construct
  MOD_RES 7: note = 4Hyp
DRDPFAXSFGAW

SEQ ID NO: 5 - protein, 12 aa, synthetic construct
  MOD_RES 7: note = 4Hyp
HYDPYAXSISTV

SEQ ID NO: 6 - protein, 12 aa, synthetic construct
  MOD_RES 9: note = 4Hyp
ASPRDPYMXSPT

SEQ ID NO: 7 - protein, 12 aa, synthetic construct
  MOD_RES 6: note = 4Hyp
DPYGGXKFHLRV

SEQ ID NO: 8 - protein, 12 aa, synthetic construct
  MOD_RES 7: note = 4Hyp
SHDPYAXSDSSV

SEQ ID NO: 9 - protein, 12 aa, synthetic construct
  MOD_RES 10: note = 4Hyp
YLPWDPYGGXQR

SEQ ID NO: 10 - protein, 12 aa, synthetic construct
  MOD_RES 11: note = 4Hyp
TPPTIRDPYAXL

SEQ ID NO: 11 - protein, 12 aa, synthetic construct
ALNLTSWPSRYA

SEQ ID NO: 12 - protein, 12 aa, synthetic construct
  MOD_RES 8: note = 4Hyp
VERDPYAXAPEY

SEQ ID NO: 13 - protein, 12 aa, synthetic construct
  MOD_RES 10: note = 4Hyp
EDVTQDPFAXTR

SEQ ID NO: 14 - protein, 12 aa, synthetic construct
  MOD_RES 12: note = 4Hyp
ASHTLMADPYAX

SEQ ID NO: 15 - protein, 12 aa, synthetic construct
  MOD_RES 12: note = 4Hyp
ANRNVYADPVKX

SEQ ID NO: 16 - protein, 12 aa, synthetic construct
  MOD_RES 7: note = 4Hyp
LLDPYAXRAWTK

SEQ ID NO: 17 - protein, 12 aa, synthetic construct
  MOD_RES 8: note = 4Hyp
THQDPYAXRLGS

SEQ ID NO: 18 - protein, 12 aa, synthetic construct
  MOD_RES 8: note = 4Hyp
SNWDPFAXRAAG

SEQ ID NO: 19 - protein, 12 aa, synthetic construct
  MOD_RES 12: note = 4Hyp
EFPTLTRDPYRX

SEQ ID NO: 20 - protein, 12 aa, synthetic construct
  MOD_RES 12: note = 4Hyp
APYWPGADPYRX

SEQ ID NO: 21 - protein, 12 aa, synthetic construct
  MOD_RES 10: note = 4Hyp
HFHTVDPFAXSG

SEQ ID NO: 22 - protein, 12 aa, synthetic construct
  MOD_RES 7: note = 4Hyp
GPDPYAXSMISN

SEQ ID NO: 23 - protein, 12 aa, synthetic construct
  MOD_RES 10: note = 4Hyp
GGSSGDPYAXSH

SEQ ID NO: 24 - protein, 12 aa, synthetic construct
  MOD_RES 12: note = 4Hyp
ASTYSSPDPYRX

SEQ ID NO: 25 - protein, 12 aa, synthetic construct
  MOD_RES 9: note = 4Hyp
VNAHDPFAXSLP

SEQ ID NO: 26 - protein, 12 aa, synthetic construct
SQDSSVTPYLLE

SEQ ID NO: 27 - protein, 12 aa, synthetic construct
TIVGRQNSTIAA

SEQ ID NO: 28 - protein, 12 aa, synthetic construct
SDIGRQNFLQAL

SEQ ID NO: 29 - protein, 12 aa, synthetic construct
SHYGVEVDLMSI

SEQ ID NO: 30 - protein, 12 aa, synthetic construct
MLEEQCQSRLSC

SEQ ID NO: 31 - protein, 12 aa, synthetic construct
DRQNEEYMRLRV

SEQ ID NO: 32 - protein, 12 aa, synthetic construct
WDPPGRQNLQHR

SEQ ID NO: 33 - protein, 12 aa, synthetic construct
PMQPGGQTRGLP

SEQ ID NO: 34 - protein, 12 aa, synthetic construct
ADCCRYIAIPGS

SEQ ID NO: 35 - protein, 12 aa, synthetic construct
GAYPSFDFRGLS

SEQ ID NO: 36 - protein, 12 aa, synthetic construct
VSNHGGRQNSFL

SEQ ID NO: 37 - protein, 12 aa, synthetic construct
DLIVGRQNTTSY

SEQ ID NO: 38 - protein, 12 aa, synthetic construct
ETKIVSLQELWI

SEQ ID NO: 39 - protein, 12 aa, synthetic construct
MGDTEQWGTGTH

SEQ ID NO: 40 - protein, 12 aa, synthetic construct
ASTCVRYQCAPL

SEQ ID NO: 41 - protein, 11 aa, synthetic construct
NPVTSSVWFHP

SEQ ID NO: 42 - protein, 12 aa, synthetic construct
NLEDRVQMRTLW

SEQ ID NO: 43 - protein, 12 aa, synthetic construct
QFGCEARACNLP

SEQ ID NO: 44 - protein, 12 aa, synthetic construct
DYWPSFDLSWED

SEQ ID NO: 45 - protein, 12 aa, synthetic construct
WGNWGLPGSTYA

SEQ ID NO: 46 - protein, 12 aa, synthetic construct
NSFDALHSVGNH

SEQ ID NO: 47 - protein, 12 aa, synthetic construct
  MOD_RES 10: note = 4Hyp
CVPTRDPYAXRM

SEQ ID NO: 48 - protein, 12 aa, synthetic construct
  MOD_RES 10: note = 4Hyp
GCPTRDPYAXRM

SEQ ID NO: 49 - protein, 12 aa, synthetic construct
  MOD_RES 10: note = 4Hyp
GVCTRDPYAXRM

SEQ ID NO: 50 - protein, 12 aa, synthetic construct
  MOD_RES 10: note = 4Hyp
GVPCRDPYAXRM

SEQ ID NO: 51 - protein, 12 aa, synthetic construct
  MOD_RES 10: note = 4Hyp
GVPKRDPYAXRM

SEQ ID NO: 52 - protein, 12 aa, synthetic construct
  MOD_RES 10: note = 4Hyp
GVPVRDPYAXRM

SEQ ID NO: 53 - protein, 12 aa, synthetic construct
  MOD_RES 10: note = 4Hyp
GVPTADPYAXRM

SEQ ID NO: 54 - protein, 12 aa, synthetic construct
  MOD_RES 10: note = 4Hyp
GVPTCDPYAXRM

SEQ ID NO: 55 - protein, 12 aa, synthetic construct
  MOD_RES 10: note = 4Hyp
GVPTDDPYAXRM

SEQ ID NO: 56 - protein, 12 aa, synthetic construct
  MOD_RES 10: note = 4Hyp
GVPTEDPYAXRM

SEQ ID NO: 57 - protein, 12 aa, synthetic construct
  MOD_RES 10: note = 4Hyp
GVPTFDPYAXRM

SEQ ID NO: 58 - protein, 12 aa, synthetic construct
  MOD_RES 10: note = 4Hyp
GVPTIDPYAXRM

SEQ ID NO: 59 - protein, 12 aa, synthetic construct
  MOD_RES 10: note = 4Hyp
GVPTRDCYAXRM

SEQ ID NO: 60 - protein, 12 aa, synthetic construct
  MOD_RES 10: note = 4Hyp
GVPTRDPYCXRM

SEQ ID NO: 61 - protein, 12 aa, synthetic construct
  MOD_RES 10: note = 4Hyp
GVPTRDPYVXRM

SEQ ID NO: 62 - protein, 12 aa, synthetic construct
  MOD_RES 10: note = 4Hyp
GVPTRDPYYXRM

SEQ ID NO: 63 - protein, 12 aa, synthetic construct
  MOD_RES 10: note = 4Hyp
GVPTRDPYAXHM

SEQ ID NO: 64 - protein, 12 aa, synthetic construct
  MOD_RES 10: note = 4Hyp
GVPTRDPYAXSM

SEQ ID NO: 65 - protein, 12 aa, synthetic construct
  MOD_RES 10: note = 4Hyp
GVPTRDPYAXRP

SEQ ID NO: 66 - protein, 12 aa, synthetic construct
  MOD_RES 8: note = 4-Methoxy-phenyalanine
  MOD_RES 10: note = 4Hyp
GVPTRDPXAXRM

SEQ ID NO: 67 - protein, 16 aa, synthetic construct
  MOD_RES 12: note = 4Hyp
DEDSYERDPYSXSQDP

SEQ ID NO: 68 - protein, 20 aa, synthetic construct
  MOD_RES 7: note = 4Hyp
  MOD_RES 14: note = 4Hyp
ERDPYSXSQDPYSXSPYDRR

SEQ ID NO: 69 - protein, 14 aa, synthetic construct
  MOD_RES 7: note = 4Hyp
  MOD_RES 14: note = 4Hyp
ERDPYSXSQDPYSX

SEQ ID NO: 70 - protein, 19 aa, synthetic construct
  MOD_RES 12: note = 4Hyp
  MOD_RES 19: note = 4Hyp
DEDSYERDPYSXSQDPYSX

SEQ ID NO: 71 - protein, 5 aa, synthetic construct
  MOD_RES 5: note = 4Hyp
DPYSX

SEQ ID NO: 72 - protein, 5 aa, synthetic construct
DPYSP

SEQ ID NO: 73 - protein, 12 aa, synthetic construct
LLDPYAPRAWTK

SEQ ID NO: 74 - protein, 16 aa, synthetic construct
  MOD_RES 12: note = 4Hyp
AEDSYERDPYSXSQDP

SEQ ID NO: 75 - protein, 16 aa, synthetic construct
  MOD_RES 12: note = 4Hyp
DADSYERDPYSXSQDP

SEQ ID NO: 76 - protein, 16 aa, synthetic construct
  MOD_RES 12: note = 4Hyp
DEASYERDPYSXSQDP

SEQ ID NO: 77 - protein, 16 aa, synthetic construct
  MOD_RES 12: note = 4Hyp
DEDAYERDPYSXSQDP

SEQ ID NO: 78 - protein, 16 aa, synthetic construct
  MOD_RES 12: note = 4Hyp
DEDSAERDPYSXSQDP

SEQ ID NO: 79 - protein, 16 aa, synthetic construct
  MOD_RES 12: note = 4Hyp
DEDSYARDPYSXSQDP

SEQ ID NO: 80 - protein, 16 aa, synthetic construct
  MOD_RES 12: note = 4Hyp
DEDSYEADPYSXSQDP

SEQ ID NO: 81 - protein, 16 aa, synthetic construct
  MOD_RES 12: note = 4Hyp
DEDSYERAPYSXSQDP

SEQ ID NO: 82 - protein, 16 aa, synthetic construct
  MOD_RES 12: note = 4Hyp
DEDSYERDAYSXSQDP

SEQ ID NO: 83 - protein, 16 aa, synthetic construct
  MOD_RES 12: note = 4Hyp
DEDSYERDPASXSQDP

SEQ ID NO: 84 - protein, 16 aa, synthetic construct
  MOD_RES 12: note = 4Hyp
DEDSYERDPYAXSQDP

SEQ ID NO: 85 - protein, 16 aa, synthetic construct
DEDSYERDPYSASQDP

SEQ ID NO: 86 - protein, 16 aa, synthetic construct
  MOD_RES 12: note = 4Hyp
DEDSYERDPYSXAQDP

SEQ ID NO: 87 - protein, 16 aa, synthetic construct
  MOD_RES 12: note = 4Hyp
DEDSYERDPYSXSADP

SEQ ID NO: 88 - protein, 16 aa, synthetic construct
  MOD_RES 12: note = 4Hyp
DEDSYERDPYSXSQAP

SEQ ID NO: 89 - protein, 16 aa, synthetic construct
  MOD_RES 12: note = 4Hyp
DEDSYERDPYSXSQDA

SEQ ID NO: 90 - protein, 12 aa, synthetic construct
  MOD_RES 7: note = 4Hyp
ALDPYAXRAWTK

SEQ ID NO: 91 - protein, 12 aa, synthetic construct
  MOD_RES 7: note = 4Hyp
LADPYAXRAWTK

SEQ ID NO: 92 - protein, 12 aa, synthetic construct
  MOD_RES 7: note = 4Hyp
LLAPYAXRAWTK

SEQ ID NO: 93 - protein, 12 aa, synthetic construct
  MOD_RES 7: note = 4Hyp
LLDAYAXRAWTK

SEQ ID NO: 94 - protein, 12 aa, synthetic construct
  MOD_RES 7: note = 4Hyp
LLDPAAXRAWTK

SEQ ID NO: 95 - protein, 12 aa, synthetic construct
LLDPYAARAWTK

SEQ ID NO: 96 - protein, 12 aa, synthetic construct
  MOD_RES 7: note = 4Hyp
LLDPYAXAAWTK

SEQ ID NO: 97 - protein, 12 aa, synthetic construct
  MOD_RES 7: note = 4Hyp
LLDPYAXRAATK

SEQ ID NO: 98 - protein, 12 aa, synthetic construct
  MOD_RES 7: note = 4Hyp
LLDPYAXRAWAK

SEQ ID NO: 99 - protein, 12 aa, synthetic construct
  MOD_RES 7: note = 4Hyp
LLDPYAXRAWTA

SEQ ID NO: 100 - protein, 5 aa, synthetic construct
  MOD_RES 5: note = 4Hyp
DYPSX

SEQ ID NO: 101 - protein, 4 aa, synthetic construct
DPYP

SEQ ID NO: 102 - protein, 16 aa, synthetic construct
  VARIANT 12: note = P can be replaced by A
DEDSYERDPYSPSQDP